(19) United States
(12) Patent Application Publication
(10) Pub. No.: US 2023/0210883 A1
(43) Pub. Date: Jul. 6, 2023

(54) COMPOSITIONS FOR THE TREATMENT OF NASH AND ASSOCIATED DISORDERS AND METHODS OF USING SAME

(71) Applicants: Duke University, Durham, NC (US); Cornell University, Ithaca, NY (US)

(72) Inventors: Phillip James WHITE, Durham, NC (US); Praveen SETHUPATHY, Ithaca, NY (US); Robert McGARRAH, Durham, NC (US)

(21) Appl. No.: 17/911,343

(22) PCT Filed: Mar. 15, 2021
(86) PCT No.: PCT/US2021/022371

§ 371 Date: Sep. 13, 2022

Related U.S. Application Data
(60) Provisional application No. 62/988,955, filed on Mar. 13, 2020.

(51) Int. Cl. A61K 31/7105; A61K 9/48; A61K 9/51

(52) U.S. Cl. CPC A61K 31/7105; A61K 9/51; A61K 9/4833; C12N 2310/141

(57) ABSTRACT

The present disclosure describes, in part, compositions and methods for the treatment of NASH and NASH-associated diseases. Compositions comprising an agent able to increases the expression, activity, or level of one more of a protective miRNA, preferably miR-375 are provided that may be used for the treatment of liver and/or liver-associated disease is treated in the subject.

### CROSS-REFERENCE TO RELATED APPLICATIONS

This application claims priority to U.S. Provisional Application No. 62/988,955 filed on Mar. 13, 2020, the contents of which are incorporated by reference in its entirety.

### STATEMENT REGARDING FEDERALLY SPONSORED RESEARCH

N/A

### SEQUENCE LISTING

A Sequence Listing accompanies this application and is submitted as an ASCII text file of the sequence listing named “155554_00594_ST25.txt” which is 594 bytes in size and was created on Mar. 12, 2021. The sequence listing is electronically submitted via EFS-Web with the application and is incorporated herein by reference in its entirety.

### BACKGROUND

Driven by the rising tide of obesity, a condition characterized by neutral lipid accumulation in the liver, termed non-alcoholic fatty liver disease (NAFLD), has rapidly become a global pandemic. It is now estimated that 1 out of 4 adults in the US has NAFLD and for approximately 1 out of every 5 cases this is accompanied by pathologic inflammation and hepatocellular damage (ballooning), termed steatohepatitis (NASH). This more pathogenic form of NAFLD progresses to fibrosis in approximately 35% of patients, significantly raising the risk for development of hepatocellular carcinoma (HCC), cirrhosis, and acute liver failure. Advanced NAFLD is also a significant risk factor for development of type 2 diabetes and cardiovascular diseases (CVD). Remarkably, there are currently no effective treatments for NASH. Moreover, the development of such agents has been hampered due to the fact that there are no sensitive plasma biomarkers that can identify NAFLD, and more importantly, distinguish between benign NAFLD and NASH.

### BRIEF SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the findings by the inventors that certain miRNAs provide a protective effect against NASH and other associated diseases, and these miRNAs can be used as a therapy for the treatment of these diseases in a subject.

Accordingly, one aspect of the present disclosure provides a method of treating a liver and/or liver-associated disease in a subject comprising, consisting of, or consisting essentially of administering to the subject a therapeutically effective amount of one or more miRNAs to the subject such that the liver and/or liver-associated disease is treated in the subject.

Another aspect of the present disclosure provides a method of treating a liver and/or liver-associated disease in a subject, comprising, consisting of, or consisting essentially of an agent that increases the expression, activity, stability or level of one more of a protective miRNA such that the liver and/or liver-associated disease is treated in the subject.

In some embodiments, the miRNA comprises miRNA-375 (SEQ ID NO:1) and/or fragments or analogues thereof.

In other embodiments, the agent is selected from the group consisting of nucleic acid molecule, a polypeptide, an antibody, a small molecule, combinations thereof, and the like.

In another embodiment, the disclosure provides a method of increasing the expression or stability of miR-375 in a liver cell, the method comprising delivering miR-375 or a fragment thereof, a miR-375 mimic, or a nucleic acid sequence encoding miR-375 to the liver cell in an amount effective to increase expression or stability of the miR-375 within the liver cell.

Another aspect of the present disclosure provides all that is described and illustrated herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a volcano plot showing that miRNA-375 is strikingly enriched in livers from NAFLD/NASH resistant severely obese persons carrying the PNPLA3 I148 polymorphism that confers genetic risk for NAFLD/NASH (CG-NASH resistant) in accordance with one embodiment of the present disclosure.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result (for example, within a +/- 10% from a given numerical value).

Unless otherwise defined, all technical terms used herein have the same meaning as commonly understood by one of ordinary skill in the art to which this disclosure belongs.

### 1. Definitions

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, the treatment is for a liver and/or liver-associated disease.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject comprises a human who is suffering from, or at risk of developing, a liver and/or liver-associated disease.

The term “analog” as used herein generally refers to compounds that are generally structurally similar to the compound of which they are an analog, or “parent” compound. Generally, analogs will retain some characteristics of the parent compound, e.g., a biological or pharmacological activity. An analog may lack other, less desirable characteristics, e.g., antigenicity, proteolytic instability, toxicity, and the like. An analog includes compounds in which a particular biological activity of the parent is reduced, while one or more distinct biological activities of the parent are unaffected in the “analog.” As applied to polypeptides, the term “analog” may have varying ranges of amino acid sequence identity to the parent compound, for example at least about 70%, at least about 80%-85%, at least about 86%-89%, at least about 90%, at least about 92%, at least about 94%, at least about 96%, at least about 98% or at least about 99% of the amino acids in a given amino acid sequence of the parent or a selected portion or domain of the parent. As applied to polypeptides, the term “analog” generally refers to polypeptides which are comprised of a segment of about at least 3 amino acids that has substantial identity to at least a portion of a binding domain fusion protein. Analogs typically are at least 5 amino acids long, at least 20 amino acids long or longer, at least 50 amino acids long or longer, at least 100 amino acids long or longer, at least 150 amino acids long or longer, at least 200 amino acids long or longer, and more typically at least 250 amino acids long or longer. Some analogs may lack substantial biological activity but may still be employed for various uses, such as for raising antibodies to predetermined epitopes, as an immunological reagent to detect and/or purify reactive antibodies by affinity chromatography, or as a competitive or noncompetitive agonist, antagonist, or partial agonist of a binding domain fusion protein function. As applied to polynucleotides, the term “analog” may have varying ranges of nucleic acid sequence identity to the parent compound, for example at least about 70%, at least about 80%-85%, at least about 86%-89%, at least about 90%, at least about 92%, at least about 94%, at least about 96%, at least about 98% or at least about 99% of the nucleic acids in a given nucleic acid sequence of the parent or a selected portion or domain of the parent. As applied to polynucleotides, the term “analog” generally refers to polynucleotides which are comprised of a segment of about at least 9 nucleic acids that has substantial identity to at least a portion of the parent. Analogs typically are at least 15 nucleic acids long, at least 60 nucleic acids long or longer, at least 150 nucleic acids long or longer, at least 300 nucleic acids long or longer, at least 450 nucleic acids long or longer, at least 600 nucleic acids long or longer, and more typically at least 750 nucleic acids long or longer. Some analogs may lack substantial biological activity but may still be employed for various uses, such as for encoding epitopes for raising antibodies to predetermined epitopes, as a reagent to detect and/or purify sequences by hybridization assays, or as a competitive or noncompetitive agonist, antagonist, or partial agonist of a target or modulator of a target.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein’s or peptide’s sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “microRNA” or “miRNA” or “miR” describes small non-coding RNA molecules, generally about 15 to about 50 nucleotides in length, for example, 17-23 nucleotides in length, which can play a role in regulating gene expression through, for example, a process termed RNA interference (RNAi). RNAi describes a phenomenon whereby the presence of an RNA sequence that is complementary or antisense to a sequence in a target gene messenger RNA (mRNA) results in inhibition of expression of the target gene. miRNAs are processed from hairpin precursors of about 70 or more nucleotides (pre-miRNA) which are derived from primary transcripts (pri-miRNA) through sequential cleavage by RNAse III enzymes. miRBase is a comprehensive microRNA database located at www.mirbase.org, incorporated by reference herein in its entirety for all purposes. In some embodiments, miRNA comprises miR-375 (also referred to as miRNA-375) and any fragments and/or analogs thereof. In some embodiments, the miR-375 comprises the sequence found in SEQ ID NO: 1 or any fragments and/or analogues thereof.

As used herein, the term “liver and/or liver-associated disease” refers to those conditions associated with non-alcoholic fatty liver disease (NAFLD), a condition associated with neutral lipid accumulation in the liver. Suitable examples of such conditions include, but are not limited to, steatohepatitis (NASH), (hepatic) fibrosis, hepatocellular carcinoma (HCC), cirrhosis, acute liver failure, hepatitis C induced NASH, and drug induced NASH, obesity-related diabetes, heart failure, clotting disorders, atherosclerosis, and the like. The FAFLD can be associated with a wide range of conditions, for example, but not limited to, fatty liver disease resulting from obesity, fatty liver disease resulting from diabetes, fatty liver disease resulting from insulin resistance, fatty liver disease resulting from hypertriglyceridemia.

By “pharmaceutically acceptable” it is meant, for example, a carrier, diluent or excipient that is compatible with the other ingredients of the formulation and generally safe for administration to a recipient thereof. As used herein, “pharmaceutically acceptable carrier” includes any material, which when combined with the conjugate retains the conjugates’ activity and is non-reactive with the subject’s immune systems. Examples include, but are not limited to, any of the standard pharmaceutical carriers such as a phosphate buffered saline solution, water, emulsions such as oil/water emulsion, and various types of wetting agents. Other carriers may also include sterile solutions, tablets including coated tablets and capsules. Typically, such carriers contain excipients such as starch, milk, sugar, some types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well-known conventional methods.

### 2. Treatment Methods

The present disclosure is based, in part, on the discovery of specific miRNAs (e.g., miR-375) play a protective role in the development of certain liver and/or liver-associated diseases associated with NAFLD, including but not limited to, steatohepatitis (NASH), (hepatic) fibrosis, hepatocellular carcinoma (HCC), cirrhosis, acute liver failure, hepatitis C induced NASH, drug induced NASH, atherosclerosis, obesity-related diabetes, heart failure, clotting disorders, and the like.

Accordingly, one aspect of the present disclosure provides a method of treating a liver and/or liver-associated disease by (a) targeting the miRNAs, increasing and/or overexpressing the miRNAs, (b) administering directly the miRNA, or (c) targeting other mRNAs that allow for the increased and/or overexpression of desired miRNAs (e.g., mir-375) as described herein. For example, in one embodiment, the present disclosure provides a method of treating a liver and/or liver-associated disease in a subject comprising, consisting of, or consisting essentially of administering to the subject a therapeutically effective amount of one or more miRNAs, an agent that increases the expression, activity, or level of one more of said miRNAs, such that the liver and/or liver-associated disease is treated in the subject. For example, in one embodiment, the method comprises administering to the subject a therapeutically effective amount of miRNA-375 (SEQ ID NO: 1) or a miR having a sequence having at least 90% identity with SEQ ID NO:1 or a fragment and/or analogue thereof, or an agent that increases the expression, level, or stability of miRNA-375. In various embodiments, the agent that modulates one or more miRNAs to treat the liver and/or liver-associated disease may comprise, consist, or consist essentially of a nucleic acid molecule, a polypeptide, an antibody, a small molecule, combinations thereof, and the like.

In one embodiment, the agent is coupled to a moiety that increases cell penetration or solubility of the agent. In one embodiment, the agent is coupled to cholesterol. In another embodiment, the agent is coupled to one or more moieties or combined with one or more compositions that are capable of directing or targeting the agent to a specific organ, tissue, or cell type. In some embodiments, the composition comprises a delivery vehicle, including but not limited to, a nanoparticle, microparticle, micelle, polymerosome, virus particle, and the like, which comprises the agent. In some embodiments, the delivery vehicle is targeted to a specific treatment site, to reduce any possible systemic effects. For example, the delivery vehicle or targeting agent may target to liver cells.

In another embodiment, the protective miRNAs are coupled to a moiety that increases cell penetration or solubility of the protective miRNA. In one embodiment, the protective miRNA is coupled to cholesterol. In another embodiment, the protective miRNA is coupled to one or more moieties or combined with one or more compositions that are capable of directing the protective miRNA to a specific organ, tissue, or cell type (e.g., the liver). Sutiable methods of targeting to the liver include, but are not limited to, for example, local delivery, liver-specific targeting of nanoparticles and liposomes, liver-specific ligand targeting, among others. For example, suitable methods of targeting the miRNA to liver cells include, but not limited to, N-acetylgalactosamine conjugation, encapsulation in lipid nanoparticles containing cholesterol, lipid based nanoparticle targeting (see, e.g., Bottger et al. “Lipid-based nanoparticle technologies for liver targeting, Advanced Drug Delivery Review, vol. 154-155, 2020 p. 79-101, incorporated by reference in its entirety regarding lipid-targeting) or conjugation with tissue specific ligands to generate liver Targeted RNAi Molecules ..

In another embodiment, the composition comprising a protective miRNA is administered locally. In another embodiment, the composition comprising a protective miRNA is administered systemically via subcutaneous or intravenous injection.

In another embodiment, one or more of the protective miRNAs or mimics thereof may be administered to a subject at risk of developing, or having been diagnosed with, or is suffering from, a liver and/or liver-associated disease as described herein. In one embodiment, the protective miRNAs administered to the subject are downregulated in the disease state. For example, in one embodiment, miR-375 is downregulated in the liver of the subject at risk of developing, having been diagnosed with or suffering from a liver or liver-associated disease described herein.

Once a subject is diagnosed with having or is at risk of having a liver and/or liver-associated disease as described herein, the subject can be treated with the miRNA and compositions and methods described herein. In another embodiment, the compositions and methods described herein can be combined with methods known in the art. Well known treatments for liver and/or liver-associated disease include, but are not limited to, drug treatments, and surgical treatments. Drug treatments used for the treatment of liver and/or liver-associated disease include, for example, statins, fibrates, anti-platelet medications, anti-coagulant medications, aspirin, lipid-lowering agents, antioxidants, bile salts, co-factors increasing the mitochondrial transport of fatty acids, and others well known in the art. Surgical treatments include, but are not limited to, gastric bypass surgery, bariatric surgery, coronary artery bypass surgery, carotid artery surgery, atherosclerosis plaque removal surgery, and atherectomy. Other treatments particularly well suited for use in the present disclosure are well known in the art. In one embodiment, the subject can be treated using dietary modification, lifestyle modification, physical therapy, or other means known in the art to treat or prevent progression of liver and/or liver-associated disease.

The effectiveness of a method or composition of the described herein can be assessed, for example, by methods known in the art for measuring the reduction is one or more characteristics associated with liver or liver associated disease. For example, measures of the efficacy of the methods of the disclosure include assessing relief of symptoms associated with fatty liver disease including, but not limited to, liver fibrosis, fat content of liver, incidence of or progression of cirrhosis, incidence of hepatocellular carcinoma, elevated hepatic aminotransferase levels, increased alanine aminotransferase (ALT), increased aspartate aminotransferase (AST), elevated serum ferritin, and cytokeratin-18 fragments. Dosage adjustment and therapy can be made by a medical specialist depending upon, for example, the severity of fatty Liver disease. For example, treatment of fatty liver disease may result in a reduction in hepatic transaminase of between approximately 10% to 40% compared to levels before treatment. In a related embodiment, treatment results in a reduction in alanine anminotransferase levels in a treated patient to approximately 30%, 20% or 10% above normal ALT levels, or at normal ALT levels (≧40 iu/L). In another embodiment, treatment with cysteamine product results in a reduction in aspartate anminotransferase levels in a patient to approximately 30%, 20% or 10% above normal AST levels or back to normal AST levels.

### 3. Modulators and Agents That Alter Expression of miRNA

The protective miRNA (miR) may be delivered via an agent, e.g., nucleic acid molecule (miR, DNA, vector, etc.), or modulated via another molecule, including, but not limited to, e.g., a nucleic acid molecules, a polypeptide, an antibody, a small molecule, combinations thereof, and the like.

### A. miRNA, Nucleic Acids and Vectors

MiRNAs are small non-coding RNA molecules that are capable of causing post-transcriptional silencing of specific genes in cells by the inhibition of translation or through degradation of the targeted mRNA. A miRNA can be completely complementary or can have a region of noncomplementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis. In some embodiments, the miRNA comprises miRNA-375 (SEQ ID NO: 1) or a sequence having at least 95% sequence similarity to SEQ ID NO:1.

In various embodiments, the agent comprises an oligonucleotide that comprises the nucleotide sequence of a protective miRNA. In certain embodiments, the oligonucleotide comprises the nucleotide sequence of a protective miRNA in a pre-microRNA, mature or hairpin form. In other embodiments, a combination of oligonucleotides comprising a sequence of one or more protective miRNAs, any pre-miRNA, any fragment, or any combination thereof is envisioned.

miRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In one embodiment, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC_{5Q}. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules may include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3′-alkylene phosphonates and chiral phosphonates, phosphinates, phosphoramidates, thionophosphoramidates, thionoalkylphosphonates, thionoalkylphosphotriest-ers, and boranophosphates. Various salts, mixed salts and free acid forms are also included.

Nucleotide oligomers having modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyl eneimino and methylenehydrazino backbones; sulfonate and sulfonamide backbones; amide backbones; and others having mixed N, O, S and CH₂ component parts. Nucleotide oligomers may also contain one or more substituted sugar moieties. Such modifications include 2′-O-methyl and 2′-methoxyethoxy modifications. Another desirable modification is 2′-dimethylaminooxyethoxy, 2′-aminopropoxy and 2′-fluoro. Similar modifications may also be made at other positions on an oligonucleotide or other nucleotide oligomer, particularly the 3′ position of the sugar on the 3′ terminal nucleotide. Nucleotide oligomers may also have sugar mimetics such as cyclobutyl moieties in place of the pentofuranosyl sugar.

In some embodiments, an miRNA as described herein is linked to a galactose trimer. As used herein, a galactose trimer comprises a molecule having three or four terminal galactose derivatives. As used herein, the term galactose derivative includes both galactose and derivatives of galactose having affinity for the asialoglycoprotein receptor equal to or greater than that of galactose. A galactose trimer contains three or four galactose derivatives each linked to a central branch point through its C-1 carbon. In some embodiments, a galactose derivative is linked to the branch point via a linker or spacer. In some embodiments, the linker or spacer is a flexible hydrophilic spacer (U.S. Pat. No. 5,885,968; Biessen et al. J. Med. Chem. 1995 Vol. 39 p. 1538-1546), such as, but not limited to: a PEG spacer. In some embodiments, the PEG spacer is a PEG3 spacer. The branch point can be any small molecule which permits attachment of three to four galactose derivatives and further permits attachment of the branch point to the miRNA agent. Attachment of the branch point to the miRNA agent may occur through a linker or spacer. In some embodiments, the linker or spacer comprises a flexible hydrophilic spacer, such as, but not limited to: a PEG spacer. In some embodiments, a PEG spacer is a PEG₃ spacer (three ethylene units). In other embodiments, the PEG spacer has 1 to 20 ethylene units (PEG₁ to PEG₂₀). In some embodiments, a galactose derivative comprises an N-acetylgalactosamine (GaLNAc or NAG). Other saccharides having affinity for the asialoglycoprotein receptor may be selected from the list comprising: galactose, galactosamine, N-formyl-galactosamine, N-acetyl-galactosamine, N-propionyl-galactosamine, N-n-butanoylgalactosamine, and N-iso-butanoylgalactosamine. The affinities of numerous galactose derivatives for the asialoglycoprotein receptor have been studied (see for example: Iobst, S. T. and Drickamer, K. J. B. C. 1996, 271, 6686) or are readily determined using methods well known and commonly used in the art. Other terms common in the art for galactose trimer having three terminal galactose derivatives include tri-antennary galactose, trivalent galactose. Other terms common in the art for galactose trimer include galactose cluster. It is known that tri-antennary galactose derivative clusters are bound to the ASGPr with greater affinity than bi-antennary or mono-antennary galactose derivative structures.

In other nucleotide oligomers, both the sugar and the internucleoside linkage, i.e., the backbone, are replaced with groups. Methods for making and using these nucleotide oligomers are described, for example, in “Peptide Nucleic Acids (PNA): Protocols and Applications” Ed. P. E. Nielsen, Horizon Press, Norfolk, United Kingdom, 1999.

In other embodiments, a single stranded modified nucleic acid molecule (e.g., a nucleic acid molecule comprising a phosphorothioate backbone and 2′-OMe sugar modifications is conjugated to cholesterol.

A miRNA described herein, which may be in the mature or hairpin form, may be provided as a naked oligonucleotide that is capable of entering a tumor cell. In some cases, it may be desirable to utilize a formulation that aids in the delivery of a miRNA or other nucleotide oligomer to cells.

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In one embodiment, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a protective miRNA, described herein. In certain embodiments, the composition comprises oligonucleotides having nucleobase identity to the nucleobase sequence of a protective miRNA, and are thus designed to mimic the activity of the protective miRNA. In certain embodiments, the oligonucleotide composition that mimics miRNA activity comprises a double-stranded RNA molecule which mimics the mature miRNA hairpins or processed miRNA duplexes.

In one embodiment, the oligonucleotide shares identity with endogenous miRNA or miRNA precursor nucleobase sequences. An oligonucleotide selected for inclusion in a composition of the present invention may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present invention encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

Treatment with a miRNA-modulating agent may be carried out using one or more nucleic acid molecules to increase the expression of preferred miRNAs (e.g., miR-375). In one embodiment, the nucleic acid comprises a heterologous promoter/regulatory sequence such that the nucleic acid is capable of directing expression of the nucleic acid. Thus, the present disclosure encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (2008, Current Protocols in Molecular Biology, John Wiley & Sons, New York) and as described elsewhere herein. In one embodiment, a vector is used to increase the level of one or more miRNAs associated with liver and/or liver-associated disease as defined herein. The term “vector,” or “recombinant vector” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”. Vectors, including expression vectors, comprise the nucleotide sequence encoding the miR or fragments thereof described herein and a heterogeneous sequence necessary for proper propagation of the vector and expression of the encoded miR. The heterogeneous sequence (i.e., sequence from a difference species than the miR) can comprise a heterologous promoter or heterologous transcriptional regulatory region that allows for expression of the polypeptide. A recombinant expression cassette comprising a polynucleotide encoding the miR or fragment thereof of the present invention is also contemplated. The polynucleotide may be under the control of a heterologous transcriptional promoter allowing the regulation of the transcription of said polynucleotide in a host cell. The present invention also provides a recombinant expression cassette comprising a polynucleotide according to the present invention under the control of a transcriptional promoter allowing the regulation of the transcription of said polynucleotide in a host cell. Said polynucleotide can also be linked to appropriate control sequences allowing the regulation of its translation in a host cell. In order to assess the expression of the mi-RNA modulating polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the present disclosure relates to a vector, comprising the nucleotide sequence of the present disclosure or the construct of the present disclosure. The choice of the vector will depend on the host cell in which it is to be subsequently introduced. In some embodiments, the vector of the present disclosure is an expression vector. Suitable host cells include a wide variety of prokaryotic and eukaryotic host cells. In some embodiments, the expression vector is selected from the group consisting of a viral vector, a bacterial vector and a mammalian cell vector. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al., and in Ausubel et al., and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

Vectors suitable for the insertion of the polynucleotides are vectors derived from expression vectors in prokaryotes such as pUC18, pUC19, Bluescript and the derivatives thereof, mp18, mp19, pBR322, pMB9, ColE1, pCR1, RP4, phages and “shuttle” vectors such as pSA3 and pAT28, expression vectors in yeasts such as vectors of the type of 2 micron plasmids, integration plasmids, YEP vectors, centromere plasmids and the like, expression vectors in insect cells such as vectors of the pAC series and of the pVL, expression vectors in plants such as pIBI, pEarleyGate, pAVA, pCAMBIA, pGSA, pGWB, pMDC, pMY, pORE series and the like, and expression vectors in eukaryotic cells based on viral vectors (adenoviruses, viruses associated to adenoviruses such as retroviruses and, particularly, lentiviruses) as well as non-viral vectors such as pSilencer 4.1-CMV (Ambion), pcDNA3, pcDNA3.1/hyg, pHMCV/Zeo, pCR3.1, pEFI/His, pIND/GS, pRc/HCMV2, pSV40/Zeo2, pTRACER-HCMV, pUB6/V5-His, pVAX1, pZeoSV2, pCI, pSVL and PKSV-10, pBPV-1, pML2d and pTDT1.

By way of illustration, the vector in which the nucleic acid sequence is introduced can be a plasmid which is or is not integrated in the genome of a host cell when it is introduced in the cell. Illustrative, non-limiting examples of vectors in which the nucleotide sequence of the present disclosure or the gene construct of the present disclosure can be inserted include a tet-on inducible vector for expression in eukaryote cells.

The vector may be obtained by conventional methods known by persons skilled in the art (Sambrook et al.). In a particular embodiment, the vector is a vector useful for transforming animal cells.

In one embodiment, the recombinant expression vectors may also contain nucleic acid molecules which encode a peptide or peptidomimetic modulator of the present disclosure, described elsewhere herein.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site, as well as protein binding domains (consensus sequences) responsible for the binding of RNA polymerase. A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, some advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. The promoters employed may be constitutive, tissue-specific, inducible, and/or useful under the appropriate conditions to direct high level expression of the introduced DNA segment. The promoter may be heterologous or endogenous.

A promoter sequence exemplified in the experimental examples presented herein is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, Moloney virus promoter, the avian leukemia virus promoter, Epstein-Barr virus immediate early promoter, Rous sarcoma virus promoter, as well as human gene promoters such as, but not limited to, the actin promoter, the myosin promoter, the hemoglobin promoter, and the muscle creatine promoter. Further, the present disclosure should not be limited to the use of constitutive promoters. Inducible promoters are also contemplated as part of the present disclosure. The use of an inducible promoter in the present disclosure provides a molecular switch capable of turning on expression of the polynucleotide sequence which it is operatively linked when such expression is desired, or turning off the expression when expression is not desired. Examples of inducible promoters include, but are not limited to, a metallothionine promoter, a glucocorticoid promoter, a progesterone promoter, and a tetracycline promoter.

Further, the present disclosure includes the use of a tissue specific promoter, which promoter is active only in a desired tissue (e.g., liver). Tissue specific promoters are well known in the art and include, but are not limited to, the albumin, alpha 1-antitrypsin, thyroxine-binding globulin, transthyretin, hepatitis B virus core protein, and hemopexin genespromoter sequences. Suitable liver-specific promoters and enhancers are described in Kramer et al. “In vitro and in vivo comparative study of chimeric liver-specific promoters” Mol Ther. 2003 Mar;7(3):375-85. doi:10.1016/s1525-0016(02)00060-6. PMID:12668133, the contents of which are incorporated by reference in its entirety. In some embodiments, the miR may be delivered via a viral vector that specifically targets liver cells. For example, a an adeno-associated virus serotype 8 (AAV8) vector may be used with one of the promoters described above, as AAV8 serotype is hepatotropic.

In other embodiments, the expression of the nucleic acid is externally controlled. For example, in one embodiment, the expression is externally controlled using the doxycycline Tet-On system.

The recombinant expression vectors may also contain a selectable marker gene which facilitates the selection of transformed or transfected host cells. Suitable selectable marker genes are genes encoding proteins such as G418 and hygromycin which confer resistance to some drugs, .beta.-galactosidase, chloramphenicol acetyltransferase, firefly luciferase, or an immunoglobulin or portion thereof such as the Fc portion of an immunoglobulin, for example, an IgG. The selectable markers may be introduced on a separate vector from the nucleic acid of interest.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene. Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the miRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Recombinant expression vectors may be introduced into host cells to produce a recombinant cell. The cells can be prokaryotic or eukaryotic. The vector of the present disclosure can be used to transform eukaryotic cells such as yeast cells, Saccharomyces cerevisiae, or mammal cells for example epithelial kidney 293 cells or U2OS cells, or prokaryotic cells such as bacteria, Escherichia coli or Bacillus subtilis, for example. Nucleic acid can be introduced into a cell using conventional techniques such as calcium phosphate or calcium chloride co-precipitation, DEAE-dextran-mediated transfection, lipofectin, electroporation or microinjection. Suitable methods for transforming and transfecting host cells may be found in Sambrook et al. (Molecular Cloning: A Laboratory Manual, 2nd Edition, Cold Spring Harbor Laboratory press (1989)), and other laboratory textbooks.

Following the generation of the miRNA polynucleotide, a skilled artisan will understand that the miRNA polynucleotide will have some characteristics that can be modified to improve the miRNA as a therapeutic compound. Therefore, the miRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio- and other modified forms of adenine, cytidine, guanine, thymine, and uridine.

### B. Polypeptides

Treatment with a miRNA-modulating agent may be carried out using one or more polypeptides. In some embodiments, the present disclosure includes an isolated peptide modulator that activates one or more miRNAs (e.g., miRNA-375) that are associated with protection against a liver and/or liver-associated disease as defined herein. In other embodiments, an isolated peptide modulator may downregulate other miRNA(s) that allow for the increased expression/activity of desired miRNAs (miR-375) that are associated with protection against a liver and/or liver-associated disease as described herein.

The variants of the polypeptides according to the present disclosure may be (i) one in which one or more of the amino acid residues are substituted with a conserved or non-conserved amino acid residue (e.g., a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The polypeptides of the present disclosure can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

A peptide modulator of the present disclosure may be conjugated with other molecules, such as proteins, to prepare fusion proteins. This may be accomplished, for example, by the synthesis of N-terminal or C-terminal fusion proteins provided that the resulting fusion protein retains the functionality of the peptide modulator.

In other embodiments, the subject peptide modulator therapeutics are peptidomimetics of the peptide modulators. Peptidomimetics are compounds based on, or derived from, peptides and proteins. The peptidomimetics of the present disclosure typically can be obtained by structural modification of a known peptide modulator sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptide inhibitors.

Moreover, as is apparent from the present disclosure, mimetopes of the subject peptides can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic.

Peptides of the present disclosure may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors.

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Antibodies and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the present disclosure may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

### C. Antibodies

The present disclosure also contemplates a modulator of a miRNA comprising an antibody, or antibody fragment, specific for at least one miRNA associated with a liver and/or liver-associated disease. In one embodiment, the antibody can activate one or more miRNAs to treat or prevent the liver and/or liver-associated disease. In other embodiments, the antibody can downregulate other miRNAs such that the expression and/or activity of a desired miRNA (e.g., miR-375) can be enhanced to treat and/or prevent a liver and/or liver-associated disease.

Methods of making and using antibodies are well known in the art. For example, polyclonal antibodies useful in the present disclosure are generated by immunizing rabbits according to standard immunological techniques well-known in the art (see, e.g., Greenfield et al., 2014, Antibodies, A Laboratory Manual, Cold Spring Harbor, N.Y.). Such techniques include immunizing an animal with a chimeric molecule comprising a portion of another molecule such as a maltose binding protein or glutathione (GSH) tag polypeptide portion, and/or a moiety such that the RNA antigen of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

One skilled in the art would appreciate, based upon the disclosure provided herein, that the antibody can specifically bind with any portion of the antigen and the full-length miRNA can be used to generate antibodies specific therefor. However, the present disclosure is not limited to using the full-length protein as an immunogen. Rather, the present disclosure includes using an immunogenic portion of the protein to produce an antibody that specifically binds with a specific antigen. That is, the present disclosure includes immunizing an animal using an immunogenic portion, or antigenic determinant, of the antigen.

Once armed with the sequence of a specific antigen of interest and the detailed analysis localizing the various conserved and non-conserved domains of the miRNA, the skilled artisan would understand, based upon the disclosure provided herein, how to obtain antibodies specific for the various portions of the antigen using methods well-known in the art or to be developed.

The skilled artisan would appreciate, based upon the disclosure provided herein, that the present disclosure includes use of a single antibody recognizing a single antigenic epitope but that the disclosure is not limited to use of a single antibody. Instead, the disclosure encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods.

Monoclonal antibodies directed against full length or peptide fragments of a protein or peptide may be prepared using any well-known monoclonal antibody preparation procedures. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the miRNA are generated from mice immunized with the miRNA using standard procedures as referenced herein.

Nucleic acid encoding the monoclonal antibody obtained using the procedures described herein may be cloned and sequenced using technology which is available in the art. Further, the antibody of the invention may be “humanized” using methods of humanizing antibodies well-known in the art or to be developed.

The present disclosure also includes the use of humanized antibodies specifically reactive with epitopes of an antigen of interest. The humanized antibodies of the present disclosure have a human framework and have one or more complementarity determining regions (CDRs) from an antibody, typically a mouse antibody, specifically reactive with an antigen of interest.

The present disclosure also includes functional equivalents of the antibodies described herein. Functional equivalents have binding characteristics comparable to those of the antibodies, and include, for example, hybridized and single chain antibodies, as well as fragments thereof.

Functional equivalents include polypeptides with amino acid sequences substantially the same as the amino acid sequence of the variable or hypervariable regions of the antibodies. “Substantially the same” amino acid sequence is defined herein as a sequence with at least 70%, at least about 80%, at least about 90%, at least about 95%, or at least 99% homology to another amino acid sequence (or any integer in between 70 and 99), as determined by the FASTA search method. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the present disclosure further include fragments of antibodies that have the same, or substantially the same, binding characteristics to those of the whole antibody. Such fragments may contain one or both Fab fragments or the F(ab′)2 fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present disclosure can be monovalent, divalent or polyvalent. Monovalent immunoglobulins are dimers (HL) formed of a hybrid heavy chain associated through disulfide bridges with a hybrid light chain. Divalent immunoglobulins are tetramers (H2L2) formed of two dimers associated through at least one disulfide bridge.

### D. Small Molecules

Treatment with a miRNA-modulating agent may be carried out using one or more small molecules. In some embodiments, treatment with such a small molecule(s) results in the increased expression and/or activity of certain miRNAs (e.g., miRNA-375) associated with a liver and/or liver-associated disease as described herein. When the modulator is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule modulator of the invention comprises an organic molecule, inorganic molecule, biomolecule, synthetic molecule, and the like.

In one embodiment, the small molecule may be H89. miR-375 expression is repressed by the protein PKA. Small molecule inhibitors, such as H89, could be used to modulate miR375 expression (see Molecular Endocrinology, Volume 26, Issue 6, 1 Jun. 2012, Pages 989-999, doi.org/10.1210/me.2011-1205, incorporated by reference in its entirety). Other small molecules that regulate PKA are contemplated in the practice if the present invention.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small molecule library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the modulators depicted here, as well as the non-salt and non-solvate form of the modulators, as is well understood by the skilled artisan. In some embodiments, the salts of the modulators of the invention are pharmaceutically acceptable salts.

Where tautomeric forms may be present for any of the modulators described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The present disclosure also includes any or all of the stereochemical forms, including any enantiomeric or diasteriomeric forms of the modulators described. The recitation of the structure or name herein is intended to embrace all possible stereoisomers of modulators depicted. All forms of the modulators are also embraced by the present disclosure, such as crystalline or non-crystalline forms of the modulators. Compositions comprising a modulator of the present disclosure are also intended, such as a composition of substantially pure modulator, including a specific stereochemical form thereof, or a composition comprising mixtures of modulator of the invention in any ratio, including two or more stereochemical forms, such as in a racemic or non-racemic mixture.

In one embodiment, the small molecule modulator of the present disclosure comprises an analog or derivative of a modulator described herein.

In one embodiment, the small molecules described herein are candidates for derivatization. As such, in some instances, the analogs of the small molecules described herein that have modulated potency, selectivity, and solubility are included herein and provide useful leads for drug discovery and drug development. Thus, in some instances, during optimization new analogs are designed considering issues of drug delivery, metabolism, novelty, and safety.

In some instances, small molecule modulators described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog”, “analogue,” or “derivative” is meant to refer to a chemical compound or molecule made from a parent compound or molecule by one or more chemical reactions. As such, an analog can be a structure having a structure similar to that of the small molecule modulators described herein or can be based on a scaffold of a small molecule modulator described herein, but differing from it in respect to some components or structural makeup, which may have a similar or opposite action metabolically. An analog or derivative of any of a small molecule modulator in accordance with the present invention can be used to treat a liver and/or liver-associated disease.

### E. Combinations

In one embodiment, the composition of the present disclosure comprises a combination of modulators described herein. For example, in one embodiment, the composition comprises an inhibitor of one or more nonprotective miRNAs disclosed herein, in combination with an agent that increases or mimics the activity of one or more protective miRNAs disclosed herein. In other embodiments, the composition comprises two or more agents that increases or mimics the activity of one or more protective miRNAs. In some embodiments, a composition comprising a combination of modulators described herein has an additive effect, wherein the overall effect of the combination is approximately equal to the sum of the effects of each agent. In other embodiments, a composition comprising a combination of modulators described herein has a synergistic effect, wherein the overall effect of the combination is greater than the sum of the effects of each individual modulator.

A composition comprising a combination of modulators comprise individual modulators in any suitable ratio. For example, in one embodiment, the composition comprises a 1:1 ratio of two individual modulators. In another embodiment, the composition comprises a 1:1:1 ratio of three individual modulators. However, the combination is not limited to any particular ratio. Rather any ratio that is shown to be effective is encompassed.

### F. Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the present disclosure may be prepared, packaged, or sold in formulations suitable for ophthalmic, oral, rectal, vaginal, parenteral, topical, pulmonary, intranasal, buccal, intratumoral, or another route of administration. Other contemplated formulations include projected nanoparticles, liposomal preparations, resealed erythrocytes containing the active ingredient, and immunologically-based formulations.

A pharmaceutical composition of the present disclosure may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the present disclosure may further comprise one or more additional pharmaceutically active agents, including, for example, chemotherapeutics, immunosuppressants, corticosteroids, analgesics, and the like.

Controlled- or sustained-release formulations of a pharmaceutical composition of the present disclosure may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intrasternal injection, intratumoral, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer’s solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion (e.g., lipid emulsions), an ion exchange resin, a sparingly soluble polymer, or a sparingly soluble salt.

A pharmaceutical composition of the present disclosure may be prepared, packaged, or sold in a formulation suitable for pulmonary administration via the buccal cavity. Such a formulation may comprise dry particles which comprise the active ingredient and which have a diameter in the range from about 0.5 to about 7 nanometers, for example, from about 1 to about 6 nanometers. Such compositions are conveniently in the form of dry powders for administration using a device comprising a dry powder reservoir to which a stream of propellant may be directed to disperse the powder or using a self-propelling solvent/powder dispensing container such as a device comprising the active ingredient dissolved or suspended in a low-boiling propellant in a sealed container. For example, such powders comprise particles wherein at least 98% of the particles by weight have a diameter greater than 0.5 nanometers and at least 95% of the particles by number have a diameter less than 7 nanometers. For example, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (e.g., having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3 butane diol, for example. Other acceptable diluents and solvents include, but are not limited to, Ringer’s solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Additionally, the molecules may be delivered using a sustained-release system, such as semipermeable matrices of solid polymers containing the therapeutic agent. Various forms of sustained-release materials have been established and are well known by those skilled in the art. Sustained-release capsules may, depending on their chemical nature, release the molecules for a few weeks up to over 100 days. Depending on the chemical nature and the biological stability of the chimeric molecules, additional strategies for molecule stabilization may be employed.

Nucleic acids may be included in any of the above-described formulations as the free acids or bases or as pharmaceutically acceptable salts. Pharmaceutically acceptable salts are those salts that substantially retain the biologic activity of the free bases and which are prepared by reaction with inorganic acids. Pharmaceutical salts tend to be more soluble in aqueous and other protic solvents than are the corresponding free base forms.

In addition to the formulations described previously, the molecules may also be formulated as a depot preparation. Such long acting formulations may be administered by implantation (for example subcutaneously or intramuscularly) or by intramuscular injection. Thus, for example, the molecules may be formulated with suitable polymeric or hydrophobic materials (for example as an emulsion in an acceptable oil) or ion exchange resins, or as sparingly soluble derivatives, for example, as a sparingly soluble salt. Alternatively, other pharmaceutical delivery systems may be employed. Liposomes and emulsions are well-known examples of delivery vehicles that may be used to deliver nucleic acids of the disclosure.

### G. Administration

One aspect of the present disclosure relates to a treatment regimen for treating or preventing a liver and/or liver-associated disease using a composition of the present disclosure. Compositions of the present disclosure may be delivered alone or in combination with other compositions of the present disclosure, and may be administered locally or systemically using appropriate methods known in the art. Administration of the compositions of the present disclosure to a subject may be carried out using known procedures, at dosages and for periods of time effective to prevent or treat a liver and/or liver-associated disease in the subject. An effective amount of the therapeutic compound necessary to achieve a therapeutic effect may vary according to factors such as the state of the disease or disorder in the subject; the age, sex, and weight of the subject.

The regimen of administration may affect what constitutes an effective amount. Further, the dosages of the compositions may be proportionally increased or decreased as indicated by the exigencies of the therapeutic or prophylactic situation. A non-limiting example of an effective dose range for a therapeutic compound of the invention is from about 1 to about 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the subject being treated, and like factors well known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

Compounds of the invention for administration may be in the range of from about 1 ug to about 10,000 mg, about 20 ug to about 9,500 mg, about 40 ug to about 9,000 mg, about 75 ug to about 8,500 mg, about 150 ug to about 7,500 mg, about 200 ug to about 7,000 mg, about 3050 ug to about 6,000 mg, about 500 ug to about 5,000 mg, about 750 ug to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, wherein the agent is a small molecule or compound, the dose of a compound of the present disclosure is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments there between.

In one embodiment, the treatment regimen comprises daily administration of a composition of the present disclosure. In one embodiment, a treatment regimen comprises administering a composition at least once daily for at least 1 day, at least 2 days, at least 3 days, at least 4 days, at least 5 days, at least 7 days, at least 10 days, at least 2 weeks, at least 3 weeks, at least 1 month, at least 2 months, at least 3 months, at least 6 months, at least 1 year or more than 1 year. In one embodiment, a treatment regimen comprises administering a composition two times daily for at least 1 day, at least 2 days, at least 3 days, at least 4 days, at least 5 days, at least 7 days, at least 10 days, at least 2 weeks, at least 3 weeks, at least 1 month, at least 2 months, at least 3 months, at least 6 months, at least 1 year or more than 1 year. In one embodiment, a treatment regimen comprises administering a composition three times daily for at least 1 day, at least 2 days, at least 3 days, at least 4 days, at least 5 days, at least 7 days, at least 10 days, at least 2 weeks, at least 3 weeks, at least 1 month, at least 2 months, at least 3 months, at least 6 months, at least 1 year or more than 1 year.

### KITS

The present disclosure also pertains to kits useful in the methods of the present disclosure. Such kits comprise components useful in any of the methods described herein, including for example, compositions for treating a liver and/or liver-associated disease, means for administering the composition, and instructional materials. Suitable compositions include, but are not limited to, miR-375, a mimic of miR-375, a nucleic acid encoding miR-375, a vector encoding and capable of expressing miR-375, a nanoparticle comprising the miR-375, mimic or nucleic acid, and agents capable of increases the expression, activity, or level of miR-375 in a cell.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Another aspect of the present disclosure provides all that is described and illustrated herein. The following Examples are provided by way of illustration and not by way of limitation.

### EXAMPLES

### Example 1: miR-375 as a Protective miR for NASH and Related Conditions

This Examples demonstrates that the inventors have identified liver targeted miR-375 as a therapy for NASH and related conditions such as but not limited to: hepatic fibrosis, HCC, hepatitis C induced NASH, drug induced NASH. The miR-375 may also be used for lowering risk of obesity-related diabetes, heart failure and clotting disorders since NASH is an independent risk factor for each of these conditions.

A unique set of 60 human liver biopsies and matched plasma samples were acquired from a well characterized bariatric surgery cohort from the Quebec Heart and Lung Institute (QHLI) Biorepository at Laval Université. 48% of this population possess at least one allele for the common variant in the gene encoding PNPLA3 which is associated with the presence of severe NAFLD and high risk of progression to fibrosis. Thus individuals with severe obesity (BMI>40) that also carry the PNPLA3 risk allele (CG) represent a population at extremely high risk for NASH.

Within this cohort, the inventors identified a unique set of five individuals who, despite this extremely high risk, do not have NASH. Remarkably four out of these five individuals also have additional significant risk factors for NASH, type 2 diabetes (T2D) or impaired glucose tolerance (IGT) . This population has been termed “CG-NASH Resistant” (CG-NR, n=5). Next, a well matched population for BMI, sex, and diabetes status (NGT/IGT/T2D) was identified that displayed the expected severe features of NASH, termed “CG-NASH Prone” (CG-NP, n=6). Relevant clinical data for these populations are displayed in Table 1.

TABLE 1

| Clinical Variable | CG-NASH Prone (n=6) | CG-NASH | P-Val |
|---|---|---|---|
| Sex (Female%) | 100% | 100% | |
| Age | 42.5±4.2 | 52.6±2.2 | 0.068 |
| BMI | 49.4±1.4 | 51.96±2.4 | |
| WHR | 0.94±0.02 | 0.96±0.03 | |
| NGT/IGTITTD | 21212 | 1/212 | |
| HbAlc | 0.066±0.005 | 0.075±0.01 | |
| Hdl (mMl | 1.335±0.69 | 1.422±0.17 | |
| Ldl (mM) | 2.76±0.22 | 2.47± 0.52 | |
| Plasma TG (mM) | 1.59±0.13 | 1.51±0.24 | |
| % Steatosis | 73.3±4.01 | 13.2±7.26 | <0.0001 |
| Steatosis (0/11213) | 0/0/1/5 | 21211/0 | <0.005 |
| Ballooning (0/11213) | 0/31210 | 510/0/0 | <0.005 |
| Lob. Inflammation (0/1121314} | 0/21211/0 | 31210/0/0 | <0.05 |
| Port. Inflammation (0/1121314} | 0/21211/0 | 510/010/0 | <0.0001 |

| | | | |
|---|---|---|---|
| Fibrosis (0/1121314} | 01511/0/0 | 411/0/0/0 | <0.0001 |
| NAS score | 7.17±0. 7 | 1.4±0.68 | <0.0005 |
| K/V(JJMJ | 16±1.18 | 12.2±1.06 | <0.05 |

*Note the newly identified metabolite marker of NASH, a-ketoisovalerate (KIV), is also significantly (P<0.05) lower in this NASH resistant population (see PCT/US2019/037746).

### CG-NASH Resistant Individuals Express Factors in the Liver That Are Protective Against NASH (and Can be Leveraged as New Therapeutics)

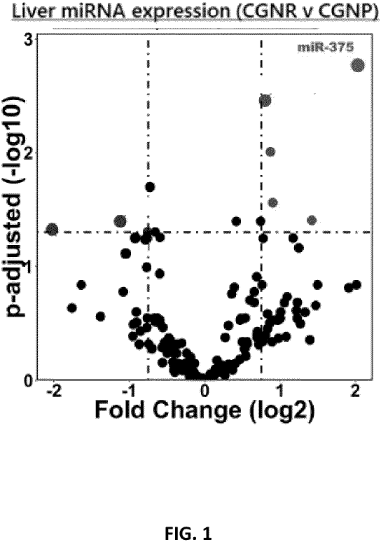

Next, the inventors performed small RNA-Seq that showed a little studied microRNA, miR-375 (CCCCGCGACGAGCCCCUCGCACAAACCGGACCUGAGCGUUUUGUUCGUUCGGCUC GCGUGAGGC (hsa-mir-375 MI0000783, SEQ ID NO: 1, www.mirbase.org/cgi-bin/mirna_entry.pl?acc=MI0000783), is strikingly enriched in livers from the CG-NASH resistant cohort (see FIG. 1). Thus, the ability to increase the amount of mir-375 in liver cells may have a protective effect on reducing or inhibiting NASH development.

miR-375 has the following targets: (i) JunD, the inflammatory transcription factor, (ii) RGS16, a liver enzyme that is activated by extracellular growth factor signaling to inhibit fatty acid oxidation. (iii) ELF, an enzyme known to participate in liver fibrosis. (iv) CTGF, the major connective tissue mitoattractant secreted by vascular endothelial cells. (v) PTDSS2, the enzyme that makes phosphatidylserine a key component of lipid droplet membranes. (vi) PRLR, the prolactin receptor promotes lipogenesis. (vii) RASD1, a gene shown to promote glucocorticoid-induced adipogenesis. (viii) ZFP36L2, a zinc-finger protein that promotes decay of mRNA for the low-density lipoprotein receptor. (ix) PTPRT, a phosphatase that promotes HF diet-induced obesity, and (x-xii) CDKN2B, FUT8, and SLC4A4, three enzymes whose induction is associated with cancer. Since the constellation of miR-375 targets outlined above cover biological pathways related to lipid metabolism, fibrosis, inflammation, and carcinogenesis, suggesting that miR-375 administration is remarkably well suited for the treatment of NASH as well as prevention of fibrosis and HCC.

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method of treating a liver or liver-associated disease in a subject comprising administering to the subject a therapeutically effective amount of a composition comprising miR-375 or a fragment thereof, a miR-375 mimic, or a nucleic acid sequence encoding miR-375 to the subject such that the liver or liver-associated disease is treated in the subject.

2. The method of claim 1, wherein the miRNA-375 comprises SEQ ID NO: 1 or a sequence with at least 90% homology to SEQ ID NO: 1.

3. The method of claim 1, wherein the composition comprises a nanoparticle capable of encapsulating the miR-375, a miR-375 mimic, or a nucleic acid sequence encoding miR-375 for administration to the subject.

4. The method of claim 3, wherein the nanoparticle comprises a targeting agent or delivery vehicle to target the miR to the liver.

5. The method of claim 1, wherein the composition comprises a vector capable of expressing the miR-375 or miR-375 mimic in a liver cell of the subject.

6. The method of claim 1, wherein the liver or liver-associated disease is selected from the group consisting of steatohepatitis (NASH), (hepatic) fibrosis, hepatocellular carcinoma (HCC), cirrhosis, acute liver failure, hepatitis C induced NASH, and drug induced NASH.

7. The method of claim 1, wherein the composition is administered locally to liver cells.

8. A method of increasing the expression of miR-375 in a liver cell, the method comprising delivering miR-375 or a fragment thereof, a miR-375 mimic, or a nucleic acid sequence encoding miR-375 to the liver cell in an amount effective to increase expression of the miR-375 within the liver cell.

9. The method of claim 8, further comprising: transducing the liver cell with a nucleic acid sequence capable of expressing the miR-375 in the liver cell.

10. The method of claim 9, wherein the nucleic acid is a vector comprising a liver-specific promoter.

11. The method of claim 10, wherein the vector is a viral vector.

12. The method of claim 8, wherein the liver cell is in vivo in a subject having liver or liver-associated disease, wherein the liver or liver-associated disease is selected from the group consisting of steatohepatitis (NASH), (hepatic) fibrosis, hepatocellular carcinoma (HCC), cirrhosis, acute liver failure, hepatitis C induced NASH, and drug induced NASH.

13. (canceled)

14. A method of treating a liver and/or liver-associated disease in a subject comprising administering an agent that increases the expression, activity, stability, or level of one more of a protective miRNA such that the liver and/or liver-associated disease is treated in the subject.

15. The method of claim 14, wherein the agent is selected from the group consisting of nucleic acid molecule, a polypeptide, an antibody, a small molecule, and combinations thereof.

16. The method of claim 15, wherein the nucleic acid molecule is a vector.

17. The method of claim 16, wherein the vector comprises a liver-specific promoter.

18. The method of claim 14, wherein the protective miRNA is miR-375 (SEQ ID NO: 1) or a mimic thereof.

19. (canceled)

20. The method of claim 14, wherein the agent is coupled to a moiety or associated with a delivery vehicle.

21. The method of claim 20, wherein the moiety or delivery vehicle increases cell penetration or solubility of the agent.

22. The method of claim 14, wherein the composition further comprises a targeting agent to target the agent to liver cells.

23-24. (canceled)


SEQUENCE LISTING (1 sequence)

SEQ ID NO: 1 - RNA, 64 nt, Homo sapiens
ccccgcgacgagccccucgcacaaaccggaccugagcguuuuguucguucggcucgcgugaggc